(19) United States
(12) Patent Application Publication
(10) Pub. No.: US 2016/0051480 A1
(43) Pub. Date: Feb. 25, 2016

(54) COMPOSITIONS AND METHODS FOR CANNABINOID COATINGS FOR USE IN DRUG DELIVERY

(71) Applicant: Medipath, Inc., Newport Beach, CA (US)

(72) Inventor: Ashraf Taha, Irvine, CA (US)

(73) Assignee: Medipath, Inc., Newport Beach, CA (US)

(21) Appl. No.: 14/834,076

(22) Filed: Aug. 24, 2015

Related U.S. Application Data
(60) Provisional application No. 62/040,613, filed on Aug. 22, 2014.

(51) Int. Cl. A61K 9/48; A61K 9/00; A61K 9/28

(52) U.S. Cl. CPC A61K 9/4891; A61K 9/288; A61K 9/0053

(57) ABSTRACT

The present invention relates to coatings and methods for the administration of cannabinoids to a patient, and in a specific embodiment, the coatings and methods may utilize or include cannabinoids, and one or more active pharmaceutical ingredients, wherein said coating is configured for oral delivery through a capsule or tablet.

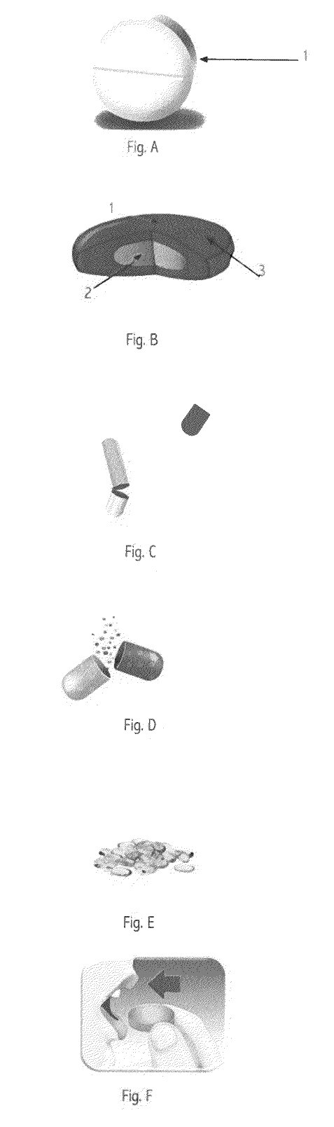

### CROSS REFERENCE TO RELATED APPLICATIONS

This application claims the benefit of and priority to U.S. Provisional Application Ser. No. 62/040,613, filed Aug. 22, 2014, the contents of which are incorporated herein by reference.

### TECHNICAL FIELD AND BACKGROUND

Many medical products and associated methods have used traditional means of drug delivery, including by way of example, oral delivery, intravenous injections, subcutaneous injections, and/or intramuscular injection. Relatedly, the cannabis plant, which is an extremely durable and malleable natural fiber, contains three different species, Cannabis sativa, Cannabis indica and Cannabis ruderalis. The present disclosure combines Cannabidiol (CBD) and other isolated cannabinoids like, for example, Cannabinol (CBN) and non-Tetrahydrocannabinol (THC) or very low THC parts of the Cannabis plant species, including resins, oils, fiber and seeds utilizing their absorptive properties used in a pill composition with Inactive or Active Pharmaceutical ingredients (APIs) providing an improved multipurpose compound for medicinal value that may be used as a coating material on pills, tablets, capsules, suppositories or any other method of medicinal delivery.

Specifically, pharmaceutical pill coatings or compositions may play a role in many medications allowing for medication to taste more pleasant, digest properly, possess time release properties, to name a few. Modernly, tablets are coated with a wide array of coatings including sugar, polymer, plasticizers and pigments. A coating composed of Cannabinoid is an attractive alternative that not only provides functionality, but is a more natural substitute to the artificial coatings in the market today. Gelatin capsules, soft and hard shell, made from bovine and animal based gelatin are considered safe despite the potential for transmittable diseases such as spongiform encephalopathy. These concerns, as well as religious and personal reasons, have led to the desire of alternative soft and hard capsule compositions.

For example, vegetable capsules are composed of Hydroxyproplymethylcellulose (HPMC) a plant polysaccharide or their derivatives like carrageenans and modified forms of starch and cellulose. These too have their limitations and are considered by some to be a processed chemical. An attractive alternative to both animal based and HPMC based capsules would be soft and hard capsules created from hemp and the cannabis species plant.

### BRIEF DESCRIPTION

By way of example and not limitation, one aspect of a composition for a cannabinoid coating is disclosed. A tablet includes an outer coating having cannabinoid, and an inner core having one or more active pharmaceutical ingredients substantially encapsulated by the outer coating.

Another aspect of a composition for a cannabinoid coating is disclosed. A capsule includes a cannabinoid outer shell, and an inner content wherein the inner content is within the cannabinoid outer shell.

One aspect of a method for facilitating the oral delivery of cannabinoids is also disclosed. The method includes providing oral delivery of cannabinoids to a patient in need thereof, wherein said method comprises administering a capsule or tablet to the patient.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

Figure A. illustrates a perspective view of a standard tablet.

Figure B. illustrates a cross section of a standard tablet with an enteric coating composed of cannabinoids and ink printing composed of cannabinoids.

Figure C illustrates a cannabinoid capsule comprised of two separate pieces that fit to make one complete cannabinoid capsule.

Figure D depicts a cannabinoid capsule releasing APIs when open or dissolved.

Figure E illustrates a soft gel capsule composed of cannabinoids.

Figure F illustrates a person about to ingest a pill having a cannabinoid coating.

### DETAILED DESCRIPTION OF ILLUSTRATIVE EMBODIMENTS

Various aspects of the illustrative embodiments will be described using terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative embodiments. However, it will be apparent to one skilled in the art that the present invention may be practiced without the specific details. In other instances, well-known features are omitted or simplified in order not to obscure the illustrative embodiments.

Various operations will be described as multiple discrete operations, in turn, in a manner that is most helpful in understanding the present invention. However, the order of description should not be construed as to imply that these operations are necessarily order dependent. In particular, these operations need not be performed in the order of presentation.

The phrase in one embodiment is utilized repeatedly. The phrase generally does not refer to the same embodiment, however, it may. The terms comprising, having and including are synonymous, unless the context dictates otherwise.

As used consistently throughout this disclosure, cannabinoids will be used herein to refer to Cannabidiol (CBD) and other isolated cannabinoids like Cannabinol (CBN) and non-Tetrahydrocannabinol (THC), or very low THC, parts of the Cannabis plant species including by way of non-limiting example Cannabis sativa (including hemp), Cannabis indica and Cannabis ruderalis and all resins, stalks, flowers, seeds and oils related thereto.

Moreover, as used consistently throughout this disclosure, the term tablet(s) will be used interchangeably and refer to inactive and active pharmaceutical ingredients formed together for drug delivery through a pill pressing technique as is known in the art of pill manufacture. Relatedly, and as used consistently throughout this disclosure, the term capsule refers to inactive and active pharmaceutical ingredients formed together for drug delivery through the capsule pinning technique as is known in the art of capsule manufacture.

Tablets and pills are a pharmaceutical dosage form. They may be defined as the solid unit dosage form of medicament or medicaments with or without suitable diluents and prepared either by molding or by compression.

Likewise, Active Pharmaceutical Ingredients (APIs) may refer to pharmaceuticals from natural origin such as plant or herbal or mineral origin, chemical drug from natural origin, drug derived from chemical synthesis, drug derived from animal origin such as hormones, drug derived from microbial origin such as antibiotics, drug derived from biotechnology genetic engineering, and drugs derived from radioactive substances.

Referring now to Figure A, Fig. A illustrates a perspective view of a standard tablet 1. Pressed Tablet or pills may be coated with many different types of coatings available on the market today. Tablet coatings include polysaccharide or polymer based, with many types of chemical components including plasticizers and pigments. Sugar coating remains a mainstay of the industry. The tablet coatings often are used for their functionality that include improving taste, eating with digestion, allowing for timed release dosage of the Active Pharmaceutical Ingredients (APIs) contained within the pill or tablet.

Providing a tablet coating that contains cannabidiol or is made from cannabinoids is an attractive option that many consumers may enjoy. Cannabinoids have many functional properties that make it ideal for inclusion in pill coatings. Additionally, the present disclosure also includes the use of cannabinoids to enterically coat medication and pills to protect the medication or pill from pH values that will decompose the pill at a rate faster than desired. Moreover, all ink used for printing on the tablets and capsules may be made from cannabinoids.

Further, there are many different types of capsules that can be referred to as soft and hard shell capsules. Traditionally, capsules are made of animal gelatin and or lactose derivative. The present disclosure consists of a natural product namely soft and hard capsules 1 that may be primarily composed of cannabinoids.

Referring now to Fig. B, Fig. B illustrates a cross section of a standard tablet 1 with a cannabinoids enteric coating 2 and cannabinoids ink printing 3 depicted. Cannabinoids possesses many physical properties that provide an ideal composition to create natural and safe soft and hard capsules 1 for drug delivery. HMPC capsules are much weaker than animal based capsules and the structural composition of cannabinoids is extremely durable and malleable. Manufacturing capsules for hard gelatin capsules uses pin molds at 22° C. that are dipped into a gelatin that is kept a temperature between 45° and 55°. After completing a series of steps and rotations the pins are stripped and the two piece capsule with a cap and body formed. The advantages of using cannabinoids as a the primary component of the soft and hard gel caps 1 is that cannabinoids have a very strong, versatile and malleable fiber. Fibers of cannabinoids have been shown to degrade when heated to temperatures higher than 160 C. °.

Creating the hard capsules will be through one of several mechanisms including pinning extrusion, injection molding, compression molding or by another technique where the final product will be composed of a capsule primarily made from cannabinoids.

Referring now to Fig. C., Figure C illustrates a cannabinoid capsule comprised of two separate pieces that fit to make one complete cannabinoid capsule. Soft gel capsules are also made of animal gelatin or non-animal derived gelatin from starch or carrageenan. The manufacturing process of soft gel caps is complicated and precise based on rotary die encapsulation process. The advantages of cannabinoids are that it possesses all the key properties to be the primary component a cannabinoids soft capsule.

Referring now to Fig. D., Figure D depicts a cannabinoid capsule releasing APIs when open or dissolved. Herein and throughout, pharmaceutical agents can refer to drugs from natural origin such as plant or herbal or mineral origin, chemical drug from natural origin, drug derived from chemical synthesis, drug derived from animal origin such as hormones, drug derived from microbial origin such as antibiotics, drug derived from biotechnology genetic engineering and drugs derived from radioactive substances. Although, as herein described, oral transport of cannabinoids is a preferred embodiment, alternative preferred embodiments may be readily apparent to a person of ordinary skill, including delivering cannabinoids orally to a patient in pill or capsule form. Figure E illustrates a soft gel capsule composed of cannabinoids that can equally be accomplished by incorporating the embodiments disclosed herein to a gel capsule manufacturing process.

While various embodiments of the disclosed technology have been described above, it should be understood that they have been presented by way of example only, and not of limitation. Likewise, the various diagrams may depict an example architectural or other configuration for the disclosed technology, which is done to aid in understanding the features and functionality that can be included in the disclosed technology. The disclosed technology is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the technology disclosed herein. Also, a multitude of different constituent module names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

1. A tablet comprising:
an outer coating having cannabinoid; and
an inner core having one or more active pharmaceutical ingredients substantially encapsulated by the outer coating.

2. The tablet of claim 1, wherein the tablet is delivered to a patient orally.

3. The tablet of claim 2, wherein the source of cannabinoid is one or more selected from the group consisting of Cannabis sativa, Cannabis indica and Cannabis ruderalis.

4. The tablet of claim 3, wherein the cannabinoid outer coating is an enteric coating.

5. The tablet of claim 4, wherein the tablet comprises different strengths and strains of cannabinoid.

6. The tablet of claim 5, wherein the enteric coating is further comprised of an active or inactive ingredient in combination with the cannabinoid.

7. The tablet of claim 5, wherein an ink is printed onto the outer coating and is further comprised of cannabinoid.

8. The tablet of claim 7, where in the ink is further comprised of an active or inactive ingredient in combination with the cannabinoid.

9. A capsule comprising:
a cannabinoid outer shell; and
an inner content wherein the inner content is within the cannabinoid outer shell.

10. The capsule of claim 9, wherein delivery of the capsule is orally.

11. The capsule of claim 10, wherein the source of cannabinoid is one or more selected from the group consisting of Cannabis sativa, Cannabis indica and Cannabis ruderalis.

12. The capsule of claim 11, wherein the capsule is a hard capsule.

13. The capsule of claim 11, where in the capsule is a soft capsule.

14. The capsule of claim 11, wherein the capsule is manufactured using a pinning process.

15. The capsule of claim 14 wherein the cannabinoid shell is an enteric coating.

16. The capsule of claim 15 wherein the inner content of the capsule can be active or inactive ingredients in combination with cannabinoids.

17. A method comprising:
providing oral delivery of cannabinoids to a patient in need thereof, wherein said method comprises administering a capsule or tablet to the patient.

18. The method of claim 17, wherein the patient is a human, and the cannabinoid is administered to the human to address one or more of Rheumatoid Arthritis, joint pain, inflammation, plantar fasciitis, migraines, muscle cramps, muscle pain, colitis, Irritable Bowel Syndrome (IBS), Post Traumatic Stress Disorder (PTSD), fibromyalgia, radiation proctitis, diaper rash, neuropathic pain, neuropathy in general, opiod tolerance, phantom pain, herpatic pain, constipation with opiods, wound care, radiation burns, amputation pain and inflammatory pain.

19. The method of claim 18, wherein the capsule or tablet is administered as needed.

20. The method of claim 19, wherein the capsule or tablet comprises different strengths and strains.
